(19) United States
(12) Patent Application Publication
(10) Pub. No.: US 2008/0097365 A1
(43) Pub. Date: Apr. 24, 2008

(54) Sealable Diaper

(76) Inventor: Louis Guthrie, Humble, TX (US)

Correspondence Address: David A. Walker;Gilbreth Roebuck Bynum Derrington, Schmidt Walker & Tran LLP, 6750 West Loop South, Suite 920, Bellaire, TX, 77401, US

(21) Appl. No.: 11/551,479

(22) Filed: Oct. 20, 2006

(51) Int. Cl. A61F 13/15

(52) U.S. Cl. 604/385.11; 604/385.01

(57) ABSTRACT

A disposable diaper includes a water impervious envelope disposed adjacent to a diaper inner layer, an opening in the envelope positioned to substantially bisect a portion of the diaper wherein the opening is opposite the inner layer and provides a seal when the envelope is inverted. The envelope may be attached to the inner layer opposite the area of the bisected portion of the diaper. The seal may be an interlocking groove and ridge, adhesive or a tape strip. The diaper may include one or more outer layers. The envelope may provide a substantial moisture barrier for the diaper. The envelope opening may be frangibly accessible through an outer layer, for example a frangible zone in the outer layer.

### BACKGROUND OF THE DISCLOSURE

### Technical Field

The disclosure is related to a diaper and, more specifically, a diaper with a sealable envelope for containing waster and sealing in odor. Disposable diapers have become a staple in millions of households. Parents now use disposable diapers on their children for the greater convenience provided over traditional cloth diapers. Disposable diapers also have large markets with the elderly and infirm.

Disposable diapers are generally made of a cloth-like waterproof exterior, a moisture-wicking inside layer, and an absorbent inner core. Disposable diapers not made of cloth have overtaken the cloth diaper market due to their convenience. The convenience of being able to throw away a soiled diaper, rather than undertake the chore of washing it, has resulted in a significant market for disposable diapers.

Although there have been advances in diaper technology, the problem still remains of what to do with a soiled disposable diaper. Soiled diapers may still present a problem, particularly when a sealed trash receptacle is not in the vicinity.

### SUMMARY

In one embodiment a disposable diaper includes a water impervious envelope disposed adjacent to a diaper inner layer, an opening in the envelope positioned to substantially bisect a portion of the diaper wherein the opening is opposite the inner layer and provides a seal when the envelope is inverted. The envelope may be attached to the inner layer opposite the area of the bisected portion of the diaper. The seal may be an interlocking groove and ridge, adhesive or a tape strip. The diaper may include one or more outer layers. The envelope may provide a substantial moisture barrier for the diaper. The envelope opening may be frangibly accessible through an outer layer, for example a frangible zone in the outer layer.

In an alternative embodiment a disposable diaper includes a water impervious envelope disposed adjacent to a diaper inner layer and an opening in the envelope positioned to substantially bisect a portion of the diaper wherein the opening is opposite the inner layer and provides a first seal when the envelope is inverted. The envelope may be attached to the inner layer in the area of the bisected portion of the diaper. The first seal is one of: i) an interlocking groove and ridge, ii) a tape strip and iii) an attached adhesive. The envelope opening may include a second seal engaged before the envelope is inverted. The diaper may include one or more outer layers. The envelope may provide a moisture barrier for the diaper. The envelope opening is frangibly accessible through an outer layer.

In still another embodiment a disposable diaper includes an inner diaper layer, an outer diaper layer, a water impervious envelope disposed adjacent to the inner diaper layer and an opening in the envelope positioned to substantially bisect a portion of the diaper wherein the opening is opposite the inner layer and provide a first seal when the envelope is inverted. The envelope may be attached to the inner layer in the area of the bisected portion of the diaper. The first seal is one of: i) an interlocking groove and ridge, ii) a tape strip and iii) an attached adhesive. The envelope opening may include a second seal engaged before the envelope is inverted. The envelope may provide a moisture barrier for the diaper. The envelope opening may be frangibly accessible through the outer layer.

### BRIEF DESCRIPTION OF THE DRAWINGS

FIG. 1 is a side view of the disposable diaper of the present invention;

FIG. 2 the mid-layer, or envelope that becomes the disposable bag;

FIG. 3 illustrates the position of an embodiment of the envelope within the diaper; and

FIG. 4 illustrates alternative positions for the envelope within the diaper.

### DETAILED DESCRIPTION

A non-limiting illustrative embodiment of a diaper is presented through one or more of its various aspects such as those noted below. Particular non-limiting embodiment related to easily isolating and sealing the interior of a diaper are described.

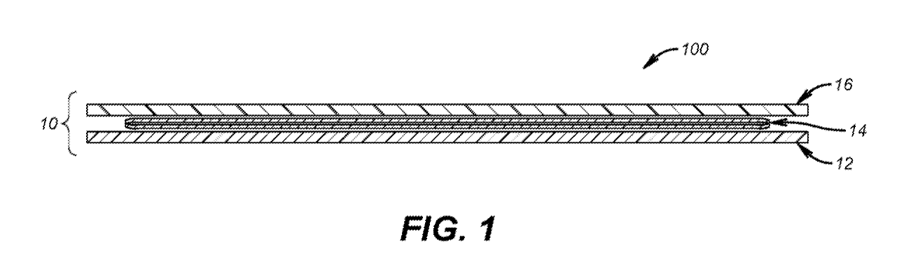

With reference to the drawings, a disposable diaper 100 is illustrated that includes at least two layers, and may include three layers. As illustrated in side-view FIG. 1, diaper 100 includes three layers. The layers are Outer layer 12, mid-layer 14 and inner-layer 16. Outer layer 12 may serve as a backing or support and other layers may be attached to outer layer 12. Outer layer 12 may be composed of a cloth-like material and be leak resistant. Layer 16, an inner layer, may be a moisture absorbent layer. Only one “inner” layer is illustrated but more are not excluded and will often be included. For example, layer 16 may include a moisture-wicking inside layer and an absorbent inner core. Additionally, an absorbent pad may by positioned adjacent an inner layer 16, for example throughout a diaper crotch area to provide more absorption, moisture wicking and/or padding. Outer layer 12 may further include a top layer (not shown), and may secure elastic leggings.

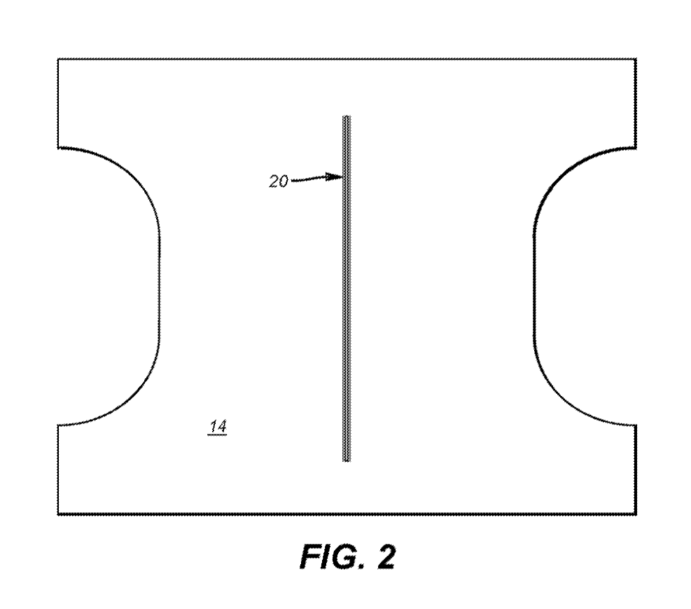

A non-limiting example of mid-layer 14 is illustrated in FIG. 2. Mid-layer 14 is an envelope or bag for enveloping the entire diaper, by inverting the envelope, prior to diaper disposal. The envelope, mid-layer 14, becomes a disposal bag wherein the contents of a soiled diaper may be sealed. The overall shape of mid-layer 14 is arbitrary, though it may be designed to provide a substantial portion of the moisture or leakage protection of the diaper. Alternatively mid-layer 14 may be designed just to provide a disposal receptacle while other layers provide moisture protection.

Mid-layer 14 has an opening 20 that may bisect the diaper, for example in the crotch area. Opening 20 is adapted to provide a moisture proof seal after the opening is opened (it may be sealed initially when positioned within the diaper), the moisture proof seal may be engaged when the mid-layer 14, that is the envelope, is inverted to enclose the diaper inner layers prior to disposal. Opening 20 does not need any particular length but may be designed to comfortable accept the soiled inner layer(s) as the mid-layer 14 envelope is inverted.

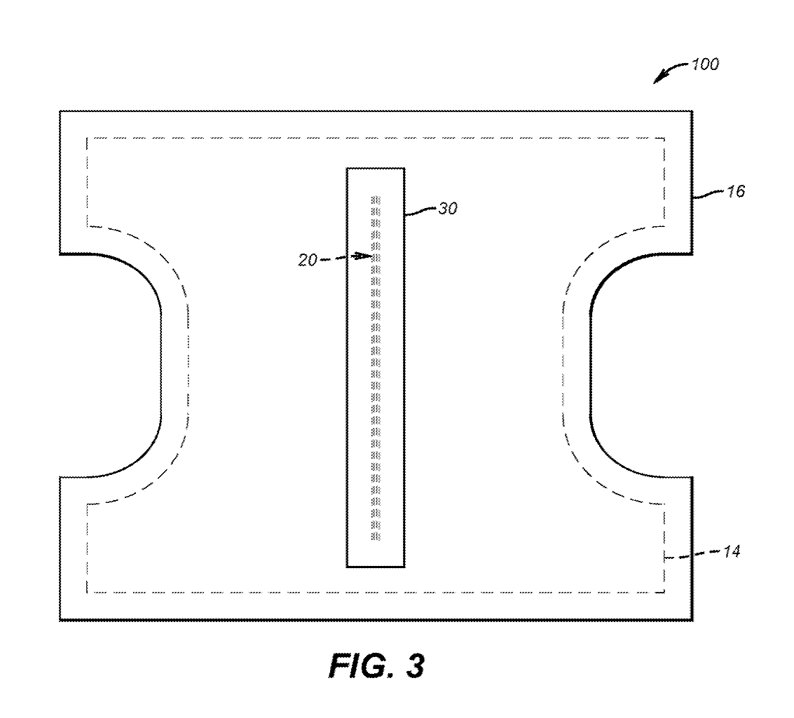

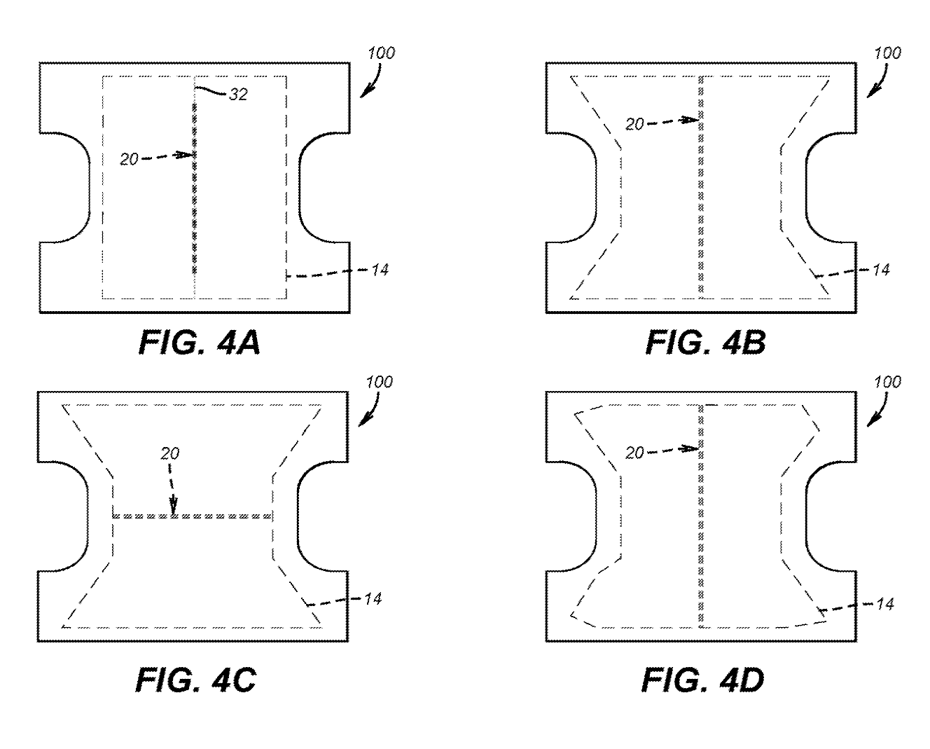

Mid-layer 14 may be detachable attached to an outer layer 12 of the diaper and may extent areally to substantially all of either or both inner layer 16 and outer layer 12 as illustrated in FIG. 3. In one embodiment mid-layer 14 may provide a substantial portion of the moisture barrier function of the diaper. In another embodiment the layer 14 envelope is not attached to inner layer 16 or only attached along a limited portion of the bisection area near the envelope opening in such a manner to facilitate pulling the inner layer(s) through opening 20. While envelope 14 can extend the length of the diaper body commensurate with outside layer 12, extending only a portion of the length may be required if envelope 14 is not fulfilling moisture barrier or protection functions (e.g., see FIG. 4A-FIG. 4D). Whether or not layer 14 is used as a primary moisture barrier, layer 14 serves a function of providing a sealable disposal bag for the inner contents of the diaper. Other non-limiting examples of mid-layer 14, the envelope that will be the diaper disposal container, are illustrated in FIG. 4. Opening 20 may bisect a portion of the diaper, but is not restricted to any particular position.

The diaper 100 is prepared for disposal by accessing opening 20 and pulling, pushing or dropping the inner diaper layers through opening 20 as or after the envelope, mid-layer 14, is inverted. Opening 20 may have been initially sealed during the construction of the diaper, for example, with a heat, adhesive or acoustic bonding, or a tape or ziplock type closure. If opening 20 is behind an outer layer 12, the outer layer may be breached or opened at the position of opening 20. A frangible zone or breachable seal (e.g., in FIG. 4A) in outer layer 12 may enable access to opening 20. Alternatively a removable tape strip attached to outer layer 12 may cover the area of opening 20. Opening 20 may then be opened; the side of mid-layer 14 opening 20 may be pulled apart to allow the inner contents of the diaper to enter into the envelope that is mid-layer 14. Opening 20 may then be sealed completely encasing the soiled inner portion of diaper for disposal.

Upon inverting the mid-layer 14 envelope about the diaper 100, the main seal in opening 20 is engaged to seal in waste and odor. Specifically, a ziplock seal or other closure mechanism may be engaged. Alternatively an adhesive tape strip, which may be present upon accessing opening 20, may enable creation of an airtight seal. The sealed diaper 100 then can be moved to any trash receptacle for disposal without the presence or an offensive odor.

Airtight interlocking closures, such as ziplock type seals may comprise a male bead mounted on one side of opening 20 and a female slot mounted on the side. Seals for opening 20 may be modified ziplock seals such that the interlocking beads have barbed profiles that firmly lock together and resist separation. Such a modified ziplock type seal prevents easy opening after being manually pressed shut.

In one embodiment a disposable diaper includes a water impervious envelope disposed adjacent to a diaper inner layer, an opening in the envelope positioned to substantially bisect a portion of the diaper wherein the opening is opposite the inner layer and provides a seal when the envelope is inverted. The envelope may be attached to the inner layer opposite the area of the bisected portion of the diaper. The seal may be an interlocking groove and ridge, adhesive or a tape strip. The diaper may include one or more outer layers. The envelope may provide a substantial moisture barrier for the diaper. The envelope opening may be frangibly accessible through an outer layer, for example a frangible zone in the outer layer.

In an alternative embodiment a disposable diaper includes a water impervious envelope disposed adjacent to a diaper inner layer and an opening in the envelope positioned to substantially bisect a portion of the diaper wherein the opening is opposite the inner layer and provides a first seal when the envelope is inverted. The envelope may be attached to the inner layer in the area of the bisected portion of the diaper. The first seal is one of: i) an interlocking groove and ridge, ii) a tape strip and iii) an attached adhesive. The envelope opening may include a second seal engaged before the envelope is inverted. The envelope opening may include a second seal engaged before the envelope is inverted. The diaper may include one or more outer layers. The envelope may provide a moisture barrier for the diaper. The envelope opening is frangible accessible through an outer layer.

In still another embodiment a disposable diaper includes an inner diaper layer, an outer diaper layer, a water impervious envelope disposed adjacent to the inner diaper layer and an opening in the envelope positioned to substantially bisect a portion of the diaper wherein the opening is opposite the inner layer and provides a first seal when the envelope is inverted. The envelope may be attached to the inner layer in the area of the bisected portion of the diaper. The first seal is one of: i) an interlocking groove and ridge, ii) a tape strip and iii) an attached adhesive. The envelope opening may include a second seal engaged before the envelope is inverted. The envelope may provide a moisture barrier for the diaper. The envelope opening may be frangibly accessible through the outer layer.

It is therefore seen that by the use of a sealable enclosure incorporated into the layers of a disposable diaper, the odor-free disposal of a soiled diaper is enabled. While various embodiments have been shown and described, various modifications and substitutions may be made thereto without departing from the spirit and scope of the disclosure herein. Accordingly, it is to be understood that the present embodiments have been described by way of illustration and not limitation.

1. A disposable diaper comprising:
a) a water impervious envelope disposed adjacent to a diaper inner layer; and
b) an opening in the envelope positioned to substantially bisect a portion of the diaper wherein the opening is opposite the inner layer and provides a seal when the envelope is inverted.

2. The disposable diaper of claim 1 wherein the envelope is attached to the inner layer opposite the area of the bisected portion of the diaper.

3. The disposable diaper of claim 1 wherein the seal is an interlocking groove and ridge.

4. The disposable diaper of claim 1 wherein the seal is a tape strip.

5. The disposable diaper of claim 1 further comprising an outer layer.

6. The disposable diaper of claim 1 wherein the envelope provides a moisture barrier for the diaper.

7. The disposable diaper of claim 1 wherein the envelope opening is frangible accessible through an outer layer.

8. A disposable diaper comprising:
a) a water impervious envelope disposed adjacent to a diaper inner layer; and
b) an opening in the envelope positioned to substantially bisect a portion of the diaper wherein the opening is opposite the inner layer and provides a first seal when the envelope is inverted.

9. The disposable diaper of claim 8 wherein the envelope is attached to the inner layer in the area of the bisected portion of the diaper.

10. The disposable diaper of claim 8 wherein the first seal is one of: i) an interlocking groove and ridge, ii) a tape strip and iii) an attached adhesive.

11. The disposable diaper of claim 8 wherein the opening further comprises a second seal that may be engaged when the envelope is not inverted.

12. The disposable diaper of claim 8 further comprising an outer layer.

13. The disposable diaper of claim 8 wherein the envelope provides a moisture barrier for the diaper.

14. The disposable diaper of claim 8 wherein the envelope opening is frangible accessible through an outer layer.

15. A disposable diaper comprising:
a) an inner diaper layer;
b) an outer diaper layer;
c) a water impervious envelope disposed adjacent to the inner diaper layer; and diaper wherein the opening is opposite the inner layer and provides a first seal when the envelope is inverted.

16. The disposable diaper of claim 15 wherein the envelope is attached to the inner layer in the area of the bisected portion of the diaper.

17. The disposable diaper of claim 15 wherein the first seal is one of: i) an interlocking groove and ridge, ii) a tape strip and iii) an attached adhesive.

18. The disposable diaper of claim 15 wherein the opening further comprises a second seal that may be engaged when the envelope is not inverted.

19. The disposable diaper of claim 15 wherein the envelope provides a moisture barrier for the diaper.

20. The disposable diaper of claim 15 wherein the envelope opening is frangibly accessible through the outer layer.
